(19) United States
(12) Patent Application Publication
(10) Pub. No.: US 2022/0370295 A1
(43) Pub. Date: Nov. 24, 2022

(54) PRESCRIPTION MEDICATION CONTROL DEVICE

(71) Applicant: Buck Maynard Houston LLC, Sheridan, WY (US)

(72) Inventor: Daniel C. Buck, Chicago, IL (US)

(73) Assignee: Buck Maynard Houston LLC, Sheridan, WY (US)

(21) Appl. No.: 17/749,656

(22) Filed: May 20, 2022

Related U.S. Application Data
(60) Provisional application No. 63/191,097, filed on May 20, 2021.

(51) Int. Cl. A61J 7/00; A61J 1/03; G16H 20/13; G16H 70/40

(52) U.S. Cl. CPC A61J 7/0076; A61J 1/035; G16H 20/13; G16H 70/40

(57) ABSTRACT

A medication control device includes a housing defining an interior of the housing and a door movable between an open position and a closed position. A carousel can be rotatably mounted within the interior of housing and is configured to receive a medication blister pack, and an actuator is configured to advance the carousel to move a blister of the blister pack to a dispensing area of the housing when operated by a user. A lock can be configured to selectively inhibit movement of the carousel is permitted. A controller is configured to, upon detecting advancement of the carousel, start a timer for a predetermined length of time and operate the lock to inhibit movement of the carousel until the predetermined length of time has elapsed. A tamper-evident seal is configured to retain the door in the closed position until broken, thereby only allowing destructive access to the medication.

### CROSS-REFERENCE TO RELATED APPLICATIONS

The present application claims priority to U.S. Provisional Application No. 63/191,097, filed May 20, 2021, the contents of which are hereby incorporated by reference in their entirety.

### BACKGROUND

The control of access to prescription medications is important to stem the abuse of prescription medications. While some prescription medications are addictive, others may have adverse side effects if taken in the improper amount and/or frequency. Careful control of when prescription medications are available to patients can help to prevent misuse of prescription medications. While pharmacists and/or patient care administrators may individually deliver prescription medication doses to patients, this requires significant personnel and logistical efforts to deliver drugs at such a frequency. Additionally, some patients may benefit from the autonomy of being in possession of the prescribed medication and being responsible for taking the medication at the prescribed intervals.

While both patients and care administrators/caregivers can benefit from the patient being in possession and responsible for taking prescription medication doses, such scenario also lends itself to potential prescription medication abuse. Unfettered patient access to the entire prescription of medication presents the opportunity for the patient to take doses without waiting for the entire prescription interval between doses. Therefore, additional solutions are needed to give patients control over prescription medication administration, while safeguarding against prescription medication abuse.

### BRIEF DISCLOSURE

This Brief Disclosure is provided to introduce a selection of concepts that are further described below in the Detailed Description. This Brief Disclosure is not intended to identify key or essential features of the claimed subject matter, nor is it intended to be used as an aid in limiting the scope of the claimed subject matter.

Embodiments of a medication control device may include a housing and a carousel may be located within the housing. The carousel may be configured to receive a blister pack of prescription medication and can be operable to advance a blister of the blister pack to a dispensing area of the housing. A lock may be configured to obstruct movement of the carousel. A controller may be configured to detect advancement of the carousel. After detection of advancement of the carousel, the controller may be configured to start a timer and operate the lock to obstruct movement of the carousel until a predetermined time has elapsed on the timer.

Embodiments of a medication control device may be configured to dispense prescription medication from a blister pack. The medication control device may include a housing defining an interior of the housing and a door movable between an open position and a closed position in which the interior of the housing is inaccessible. A carousel may be rotatably mounted within the interior of the housing and can be configured to receive the blister pack. An actuator may be configured to advance the carousel to move a blister of the blister pack to a dispensing area of the housing when operated by a user. A lock may be configured to selectively inhibit movement of the carousel, the lock configured to selectively move between a locked position in which movement of the carousel is obstructed and an unlocked position in which movement of the carousel is permitted. A controller may be configured to, upon detecting advancement of the carousel, start a timer for a predetermined length of time and operate the lock to inhibit movement of the carousel until the predetermined length of time has elapsed. A tamper-evident seal may be configured to retain the door in the closed position until broken, thereby only allowing destructive access to the interior of the sealed housing

Various other features, objects, and advantages will be made apparent from the following description taken together with the drawings.

### BRIEF DESCRIPTION OF THE DRAWINGS

The present disclosure is described with reference to the following Figures.

FIG. 1 is a front perspective view of an example of a medication control device as described herein.

FIG. 2 is a rear perspective view of the device of FIG. 1.

FIG. 3 is a cross-sectional schematic view of the device of FIG. 2

FIG. 4 is a front perspective view of another example of a medication control device with a closed door.

FIG. 5 is a front perspective view of the device of FIG. 4 with an open door.

FIG. 6 is a cross-sectional schematic view of the device of FIG. 5

FIG. 7 is a detailed view of the locking mechanism of the device of FIG. 6.

FIG. 8 is a detailed cross-sectional view of another example of a locking mechanism for a medication control device.

FIG. 9 is a detailed cross-sectional view of a further example of a locking mechanism for a medication control device.

### DETAILED DISCLOSURE

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Unless otherwise specified or limited, the phrases “at least one of A, B, and C,” “one or more of A, B, and C,” and the like, are meant to indicate A, or B, or C, or any combination of A, B, and/or C, including combinations with multiple instances of A, B, and/or C. Likewise, unless otherwise specified or limited, the terms “mounted,” “connected,” “linked,” “supported,” and “coupled” and variations thereof are used broadly and encompass both direct and indirect mountings, connections, supports, and couplings. Further, unless otherwise specified or limited, “connected” and “coupled” are not restricted to physical or mechanical connections or couplings.

As used herein, unless otherwise limited or defined, discussion of particular directions is provided by example only, with regard to particular embodiments or relevant illustrations. For example, discussion of “top,” “bottom,” “front,” “back,” “left” or “right” features is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally, use of the words “first,” “second”, “third,” etc. is not intended to connote priority or importance, but merely to distinguish one of several similar elements or machines from another.

Disclosed herein are examples of a medication control device configured for prescription medication that is exemplarily contained within a blister pack 12. Examples of a blister package and a method and apparatus for filling blister packaging are provided in US Patent Application Publication No. 2014/0230376 and U.S. Pat. No. 6,318,051, which are incorporated by reference herein in their entirety. The blister pack 12 includes a plurality of blisters 20 formed into a deformable plastic sheet. The plastic sheet is exemplarily backed by a foil backing that defines a blister pouch to the interior of the blister 20 within which a medicinal pill 22 (or pills) is contained. The blister pack 12 is exemplarily filled with the medication by the drug manufacturer. In an example, the blister pack 12 is only filled with one kind of medicinal pill 22, although it is recognized that examples of the blister pack 12 may not be so limited to pills of a single type.

In some embodiments, a medication control device may be configured as a reusable device that medication is loaded into by a physician, pharmacist, and/or any other clinician. Other embodiments, however, may be configured as a disposable device that is either preloaded with pills by a medication manufacturer, or the medication control device is loaded by the physician, pharmacist, and/or another clinician. A locking mechanism within the medication control device can limit the frequency at which the medication is dispensed from the device.

The present inventor has recognized that existing medication dispensing systems include overbearing security features that may make some patients feel as if they lack independence, or that their autonomy has been stripped away. This belief may be counterproductive to the patient's mental health, may lead to a patient not taking their medication as prescribed, and/or may contribute to other negative clinical outcomes. Whereas existing medication dispenser devices seek to limit a patient's ability to access the medication by adhering to a rigid daily medication schedule, sometimes with automated dispensing, embodiments of the present medication control device limit the frequency at which medication can be dispensed from the device while still allowing the patient to take the medication according to their own schedule. This may be useful, for example, in fostering the patient's sense of independence and responsibility.

The inventor has further recognized that, without constant supervision, an individual who is determined to access their medication outside of the prescribed dose schedule is likely to be successful in doing so. Rather than attempting to eliminate a user/patient's ability to get to the medication contained within, the present medication control device may be equipped with limited security features or barriers to unauthorized access to the medication. In the illustrated embodiments, for example, the medication control device includes tamper-evident security features that allow the user to destructively access the medication within the device, but that alert the physician and/or pharmacist if the user does so. This presents patients with a clear choice: take the medication as prescribed or risk losing access to the medication altogether. Giving the patient this responsibility can empower them, further building a sense of autonomy and responsibility. The medication control device may also be useful in situations where intentional abuse of the medication is not a risk, such as a patient with reduced mental faculties or a degenerative disease. For example, an Alzheimer's or dementia patient can use the device to control their own medication while helping to prevent accidental overdoses. A sense of losing one's autonomy has been linked to a decline in Alzheimer's and dementia patients' overall health and an increase in negative clinical outcomes. Thus, the present inventor has recognized a need for a medication control device that allows patients to safely access their medication while maintaining their independence and fostering responsibility.

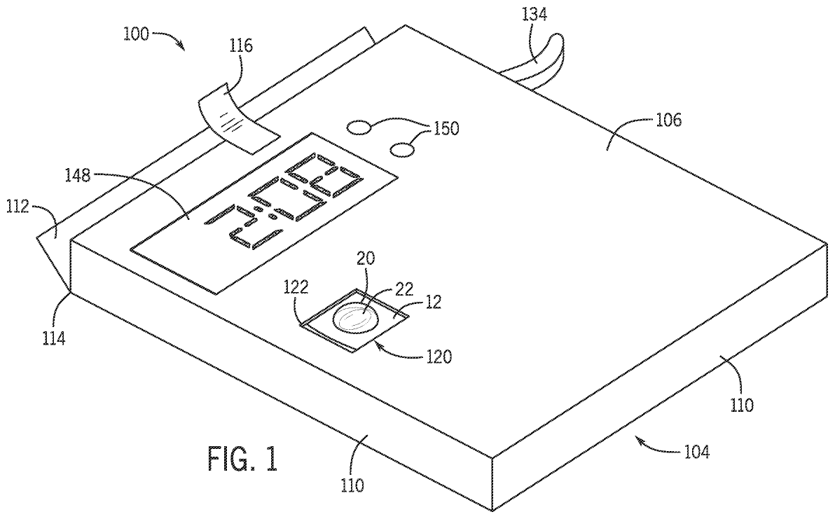

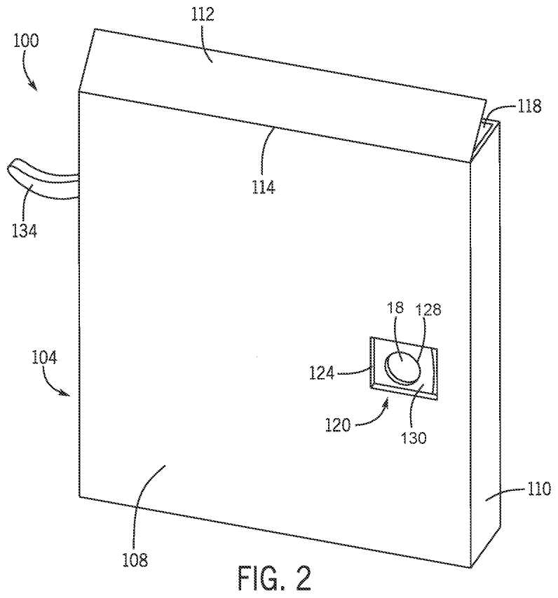

Referring to FIGS. 1 and 2, an embodiment of a prescription medication control device 100 is illustrated. The medication control device 100 is exemplarily in the form of a box or a case defining an outer housing 104. The housing 104 includes a front face 106 opposite a back face 108 and further includes side walls 110 that connect the front face 106 to the back face 108 and exemplarily give the outer housing a depth dimension. At least one of the length, depth, and height dimensions of the housing 104 may be selected based upon the dimensions of the blister pack 12 configured to be received in the device 100. In a merely exemplary and non-limiting description, the front face 106 (and similarly the back face 108) may be approximately 6 in. by 7 in. while the side walls 110 are between 0.25-0.5 in. It will be recognized that these dimensions are merely exemplary and are not limiting on the scope or size of the medication control device as described herein. In some embodiments, for example, the housing of a medication control device has at least one of a different length, depth, and height dimension, and/or the housing may be a different shape, such as a cylindrical housing, a triangular housing, or any other shape.

The medication control device 100 further includes a door 112, which is configured to rotate on a hinge 114 and exemplarily provides a top sidewall of the outer housing 104. In other embodiments, a medication control device 100 may include a door that provides or is a part of a different one of the side walls 110, and/or the door 112 may extend partially along at least one of the front face 106 and the back face 108. In a still further example, the outer housing 104 may be in the form of a clamshell design with a door hinge exemplarily located between the back face 108 (or front face 106) and one of the side walls 110. The door 112 is movable between an open position and a closed position. When the door 112 is in the open position, a patient can access the interior 126 of the housing through the opening 118, whereby the housing 104 can be loaded with a blister pack 12 of the prescribed medicinal pills 22. When in the closed position, the door 112 prevents access to the housing 104 through the opening 118, thereby making the interior 126 of the housing 104 inaccessible to the patient and preventing the blister pack from being removed.

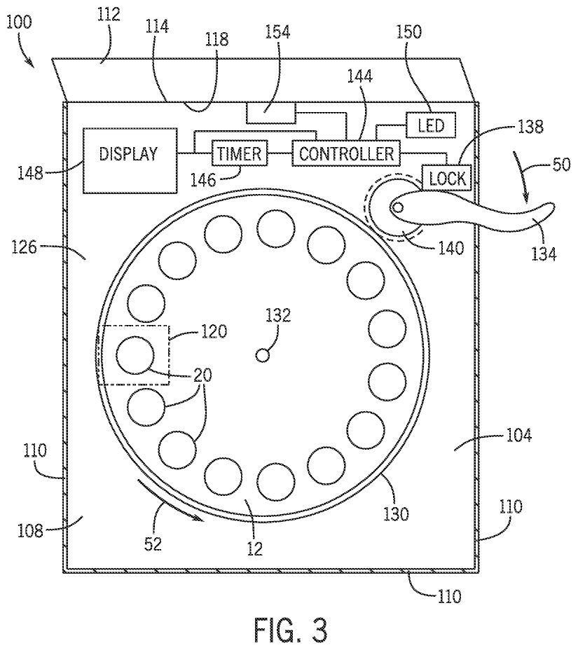

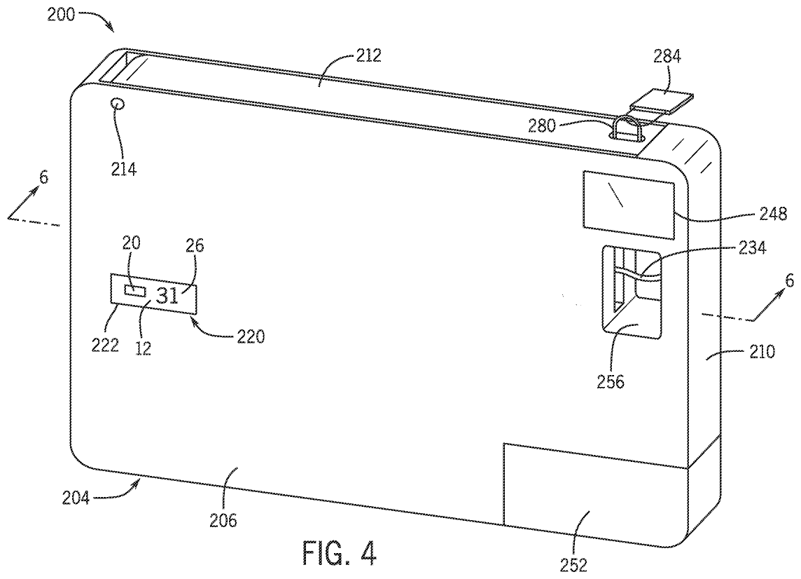

Referring to FIG. 3, the medication control device 100 may be configured to receive the blister pack 12 on a carousel 130. In the illustrated embodiments, the carousel 130 is rotatably mounted on a shaft 132 that extends from the back face 108 of the housing 104. Some embodiments, however, may include a carousel that is secured to a different part of the housing. Rotation of the carousel 130 is controlled by an actuator that extends outside of the housing 104 so that is accessible to the patient. In the embodiments of FIGS. 1-3, the actuator is configured as a lever 134 that extends through a lateral side wall 110 of the housing 104. Other embodiments, however, may have a differently configured actuator or lever (see, e.g., FIGS. 4-9). The lever 134 is operatively linked to the carousel 130 by a transmission 140, which may include at least one of a gear, a ratchet, and any other mechanical components. Movement of the lever 134, by a pushing or a pulling motion for example, in the direction of arrow 50 operates the transmission 140 to transfer the movement of the lever 134 to rotation of the carousel 130 in the direction of arrow 52. In the illustrated embodiments, the transmission 140 is configured as a ratcheting transmission 140 that allows the lever 134 to return to its original position by moving in a direction opposite the arrow 50 without causing reciprocal movement of the carousel 130.

Rotation of the carousel 130 with actuation of the lever 134 advances the carousel 130 and the blister pack 12 connected to the carousel 130 in the direction of arrow 52 by an increment of one blister 20 of the blister pack 12, thereby moving a blister 20 into a dispensing area 120 of the housing 104. In the illustrated embodiment, the dispensing area 120 includes a front window 122 formed in the front face 106 of the housing 104 and a back window 124 formed in the back face 108. The front window 122 and the back window 124 are exemplarily aligned, for example, such that a blister 20 of the blister pack 12 is visible and accessible through the front window 122 while a corresponding portion of the foil backing 18 is visible and accessible through the back window 124. In operation, when a blister 20 is positioned at the dispensing area 120, the patient can push against the blister 20 through the front window 122 to break the pill(s) 22 through the foil backing 18 to dispense the pill(s) 22 through the back window 124, thereby dispensing the pill(s) 22 from the blister pack 12 and the device 100. Some embodiments may include a differently configured dispensing area. For example, the back window may not be aligned with the front window in order to increase the difficulty of unauthorized access to the medication.

In some embodiments, the prescription medication control device 100 a carousel 130 may be dimensioned such that a diameter of the carousel is larger than the diameter of the blister pack 12. In such an embodiment, the carousel 130 may include openings so that the pill(s) can be pushed through the body of the carousel 130 to the back window (124). In the illustrated embodiments, for example, a plurality of carousel windows 128 (see FIG. 2) are formed around the carousel 130 proximate a circumferential edge thereof. Each carousel window 128 corresponds to one of the blisters 20 on the blister pack 12, and the carousel windows 128 are arranged such that each carousel window 128 is aligned with the corresponding blister 20. This may be useful, for example, so that the front face of the carousel 130 can serve as a support surface for the blister pack 12 as a patient presses the pill(s) 22 through the foil backing 18.

Some embodiments of the carousel may be configured differently than those of the illustrated embodiments. For example, the carousel may include at least one opening with a different shape and/or size than at least one other opening formed through the carousel. A carousel may include at least one slot extending radially inward from the perimeter of the carousel so that the medication in a blister corresponding to the slot can be passed through said slot. Further still, some embodiments of a medication control device may include a carousel that is dimensioned such that the diameter of the carousel is small enough that the body of the carousel does not block the medication from getting to the back window when dispensed.

Once the housing 104 is loaded with the blister pack 12, the housing 104 is closed by moving the door 112 into the closed position. Some embodiments of a prescription medication control device 100 may include at least one mechanical interlock feature configured to retain the door 112 in the closed position. For example, As illustrated in FIG. 1, the medication control device 100 may include a tamper-evident seal, such as an adhesive security tag 116, configured to hold the door closed. The adhesive security tag 116 is adhered to the door 112 and at least one of the front face 106, the back face 108, and a side wall 110, to seal the device and prevent the door 112 from opening without irreversibly damaging or destroying the adhesive security tag 116. The adhesive security tag 116 is constructed such that it will be readily apparent if the adhesive security tag 116 has been tampered with to gain access to the interior 126 of the housing 104, and the blister pack 12 within. The medication control device may include at least one different type of tamper-evident seal or security tag, and/or a security device or seal that does not provide evidence of tampering.

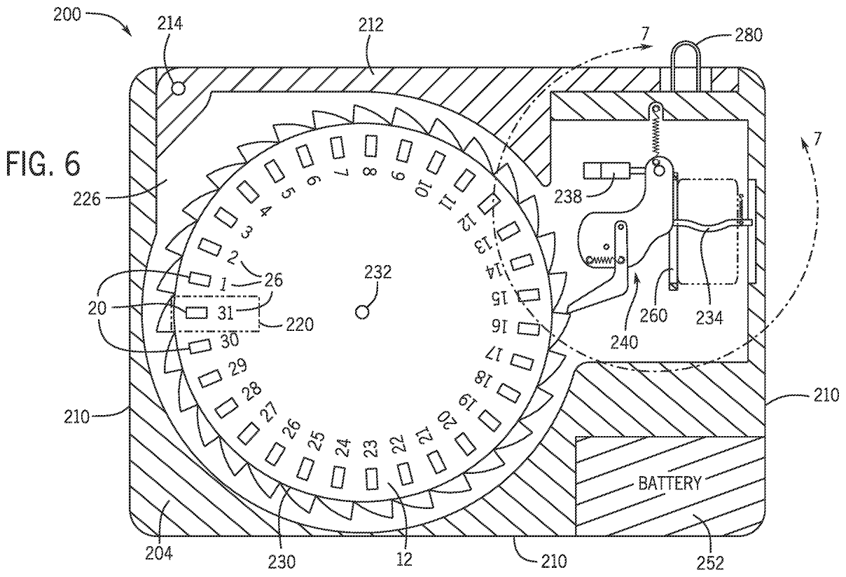

Embodiments of a medication control device 100 may include features that limit the frequency at which medication can be dispensed, and/or features that provide various indicators and information to the patient, pharmacist, and/or physician. For example, referring to FIGS. 1 and 3, the medication control device 100 includes features that, after a dose of medication is dispensed from the device 100, limit premature access to the remaining prescription medication. Within the housing 104, the medication control device 100 may include a controller 144 configured to operate an electromechanical lock 138 to selectively inhibit movement of the lever 134 and/or rotation of the carousel 130. The controller 144 is configured to determine when a dose of medication has been dispensed from the device 100 by detecting at least one of rotation of the carousel 130 and movement of the lever 134. At least one sensor integrated into the controller 144 and/or a sensor communicatively connected to the controller 144 may be used to detect movement of the carousel 130 and/or the lever 134. Upon the detection of a dispense event, the controller 144 may start a timer 146 set to run for a predetermined length of time while controlling the lock 138 to prevent another blister 20 from being advanced to the dispensing area 120. In the illustrated embodiment, the electromechanical lock 138 is configured to mechanically obstruct the lever 134 from being moved and prevent the carousel 130 from advancing. It will be recognized that such obstruction may be physically carried out by obstructing the movement of at least one of the carousel 130, the transmission 140, and the lever 134. In some embodiments, the electromechanical lock 138 may be a locking pin that is actuated between a locked position and an unlocked position by an electric motor (see, e.g., FIGS. 6-9). Once the predetermined amount of time has elapsed, the controller 144 can control the electromechanical lock 138 to move into the unlocked position so that another dose of medication may be dispensed from the device.

In the illustrated embodiment, the timer 146 is a separate circuit component that is communicatively connected to the controller 144. Some embodiments, however, may be configured to implement the timer on the controller 144. The predetermined amount of time may be a minimum dose interval for the medication in the blister pack 12, which is the minimum time that a patient should wait before taking a subsequent dose of the medication. This may be useful, for example, to prevent a patient from taking too much medication, accidentally or otherwise. The predetermined amount of time may be preprogrammed in the timer 146 and/or the controller 144, or it may be set and set by the pharmacist or physician providing the medication control device 100 to the patient. For example, the medication control device 100 may be constructed in a single-use configuration whereby the minimum dose interval is established by the manufacturer at the, for example, along with securing the blister pack 12 within the housing 104. In another embodiment, the medication control device 100 may be configured for multiple uses, in which a pharmacist may be able to replace the blister pack 12 within the housing 104, and the controller 144 may be reprogrammable to set a different minimum dose interval.

As previously mentioned, the medication control device 100 may further include information-providing features. With continued reference to FIGS. 1 and 3, the medication control device 100 may further include a display 148, for example, a liquid crystal display (LCD) that visually presents the remaining duration in the prescription interval time. While the timer 146 is counting down, the lock 138 remains engaged to prevent further advancement of a blister 20 filled with a prescription pill 22 to the dispensing area 120. The controller 144 may be configured to control at least one indicator light 150, for example, an LED light, to provide a further indication of whether the prescription interval time has elapsed, and a subsequent pill can be dispensed. The controller 144 may illuminate an indicator light 150 in one color (e.g., red) to indicate that the prescription interval time is counting down, or the indicator light 150 may be illuminated in another color (e.g., green) to indicate that the interval time has elapsed and that the patient may operate the lever 134 to advance the carousel 130 and dispense another dose of the prescription medication.

In addition to a tamper-evident seal, some embodiments of the medication control device 100 may include electronic security features. For example, the controller 144 may be in electrical communication with a tamper sensor 154 configured to determine if the door 112 has been opened to gain access to the interior 126 of the housing 104. In some embodiments, the adhesive security tag 116 may complete an electrical circuit between two terminals on the tamper sensor or within the housing 104, and thus disruption or disconnection of the security tag 116 can be sensed, identified, and indicated, for example by illumination of an indicator light 150 or providing a tamper indication on the display 148. A tamper sensor 154 may be configured to detect the opening of the door 112 without interacting with the security tag 116, thereby providing an independent indication of tampering.

It will be recognized that the medication control device 100 further includes a single-use, rechargeable, and/or replaceable battery (see, e.g., FIGS. 4-9) that provides power to the system and components as described herein. In a still further example, electrical power, for example from the battery, may be used to operate a motor (not depicted) configured to advance the carousel 130, actuated upon a push button or other user input device as may be used herein, rather than actuation of a lever by a patient to provide physical movement of the carousel 130. In a still further exemplary embodiment, the dispensing area 120 may move relative to the rest of the housing 104 and/or the blister pack 12 such as to position the dispensing area 120 relative to a subsequent blister 20 rather than the example previously described wherein the blister 20 was advanced to the dispensing area 120.

FIGS. 4-9 depict another example of a medication control device 200. It will be recognized that similar reference numbers may refer to similar components between the examples or that components described with respect to one example may be incorporated or combined with another example, while remaining within the scope of the present disclosure. The medication control device 200 may include an actuator that is internal to the housing 204. The medication control device 200 includes a trigger 234 mounted within a trigger window 256 formed into the housing 204. The trigger window 256 may extend through both the front face 206 and the back face 208 of the housing 204, as depicted the illustrated embodiments, or the trigger window 256 may be formed in one of the front face 206 or the back face 208 and extend only partially through the housing 204. To power the electronics of the medication control device 200, a battery may be housed in a battery compartment 252. The battery compartment 252 may be accessible without opening the door 212, as depicted in the embodiments of FIGS. 4-9, or the battery may be alternatively accessible through the opening 218 when the door 212 is open. As a tamper prevention measure, the controller (not shown) of the medication control device 200 may be configured to determine if the battery has been removed or if power supply to the device electronics is otherwise disrupted. If a power disruption is detected, the controller may provide a power disruption and/or tamper indication on the display 248 and/or an indicator light (not shown). A backup energy storage, for example, a battery or a capacitor may provide power to limited systems in the event of a power disruption.

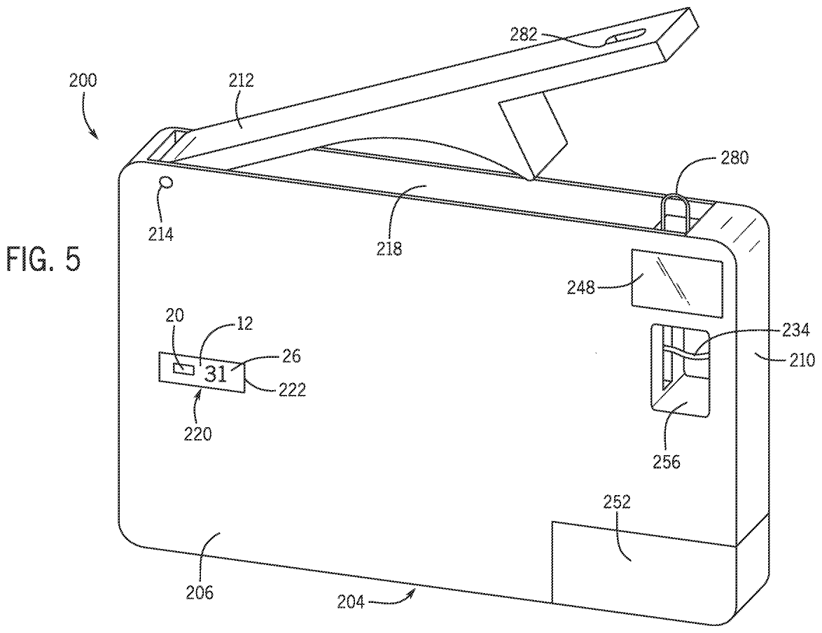

As illustrated in FIGS. 4 and 5, the door 212 may be configured to pivot around a hinge 214 extending between the front face 206 and the back face 208 and may include a slot 282 configured to receive a shackle 280, exemplarily configured as a U-shaped shackle extending from a top side of the device 200. When the door 212 is in the closed position, the shackle 280 may extend through the slot 282 so that a lock can be used to secure the door 212 in the closed position. As illustrated in FIG. 4, the lock may be configured as a tamper-evident lock 284. The tamper-evident lock 284 may be used in addition to or in place of the adhesive security tag 116 of FIGS. 1-3. In some embodiments, the tamper-evident lock 284 may include a “U” shaped loop with protruding wings configured to engage a locking block. Once inserted into the locking block, the protruding wings secure the locking tag loop to the locking block. The loop cannot be removed from the locking block without damaging the locking block, the loop, or the tag loop of the medication control device. Tamper with or damage to of any of these three components is readily apparent and will notify a pharmacist of tampering or misuse when a refill is requested.

Embodiments of the medication control device may be configured for use with blister packs 12 with different amounts of medication. For example, as illustrated in FIG. 6, the carousel 230 is configured to receive a blister pack 12 with thirty-one individual blisters 20, each one containing a dose of medication. Dose count indicators 26 may be printed adjacent to each of the blisters 20 and can indicate how many doses of medication remain in the blister pack 12. In such an embodiment, the front window 222 of the dispensing area 220 may be dimensioned so that the blister 20 and the corresponding dose count indicators 26 are visible to the patient. The controller may record the number of doses that have been dispensed from the device 200 and can use the dispense records to determine how many doses are left and display the remaining doses on the display 248.

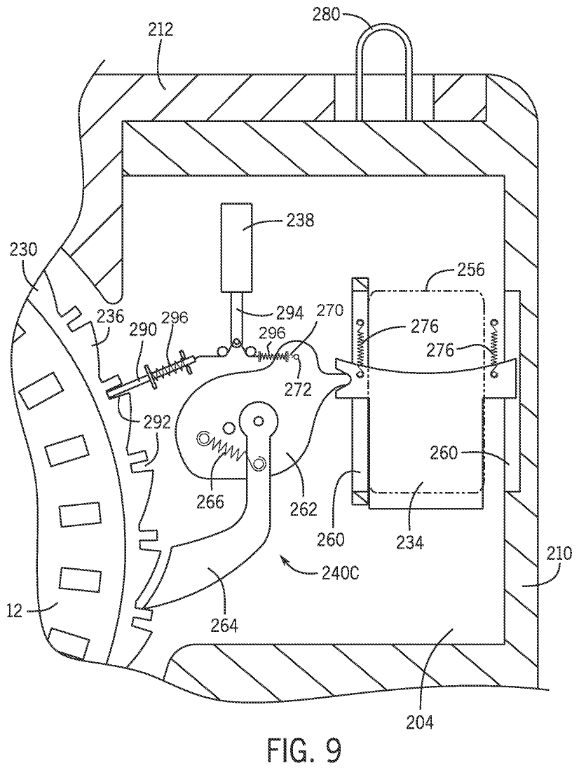

The medication control device 200 may include a carousel 230 and transmission 240 that are configured so that the carousel rotates by the appropriate amount to advance from one blister 20 to the adjacent blister. Referring to FIGS. 5 and 6, the medication control device 200 includes a ratcheting transmission 240 that is configured to advance the carousel 230 by pressing against one of the ratcheting teeth 236 formed around the perimeter of the carousel 230. The trigger 234 is mounted on rails 260 along which the trigger 234 may slide when pressed by the patient/user. The trigger 234 is mechanically linked to a connecting member 262 of the transmission 240, the connecting member 262 being movably mounted within the housing 204. In some embodiments, the connecting member 262 may be slidably mounted so that the connecting member 262 moves downward with the trigger 234 when pressed. Some examples may include a connecting member 262 that is rotatably mounted so that the connecting member 262 rotates when the trigger 234 is moved (see, e.g., FIG. 9). Further still, some embodiments of a medication control device 200 may include a transmission with at least one magnetic or electromechanical component configured to operatively link the trigger 234 (or any other actuator) to the carousel 230.

With continued reference to the figures, a ratcheting arm 264 is rotatably secured to the connecting member 262 and extends to a distal end of the arm 264 that engages the ratcheting teeth 236 of the carousel 230. When the connecting member 262 slides, rotates or is otherwise moved by the trigger 234, the distal end of the ratcheting arm 264 presses against a flat face of one of the teeth 236, thereby rotating the carousel 230 and advancing the next blister 20 of the blister pack to the dispensing area 220. After the trigger 234 is released, return springs 276 connected to at least one of the trigger 234 and the connecting member 262 bias the trigger 234 and the connecting member 262 back into their resting positions. As the connecting member 262 returns to its original position, an arm return spring 266 extending between the connecting member 262 and the ratcheting arm 264 holds the distal end of the arm 264 in abutment with the ratcheting teeth 236 without causing reciprocal rotation of the carousel 230. Some embodiments, however, may include a differently configured transmission for converting translational and/or rotational movement of the trigger 234 to rotational movement of the carousel 230.

To limit the frequency at which medication can be dispensed from the medication control device 200, a lock 238 is configured to selectively prevent movement of the connecting member 262. When a patient is prohibited from dispensing another dose of medication, the controller instructs the lock 238 to move a locking pin 270 into a locked position in which the pin 270 is engaged with a corresponding opening 272 formed in the connecting member 262. While the locking pin 27 is engaged with the opening 272, movement of the connecting member 262 is blocked, thereby preventing the trigger 234 from being pressed and the carousel 230 from being advanced. Once the predetermined amount of time has expired, the controller can instruct the lock 238 to move into an unlocked position by retracting the locking pin 270, and disengaging the pin 270 from the opening 272 so that the connecting member 262 can be moved.

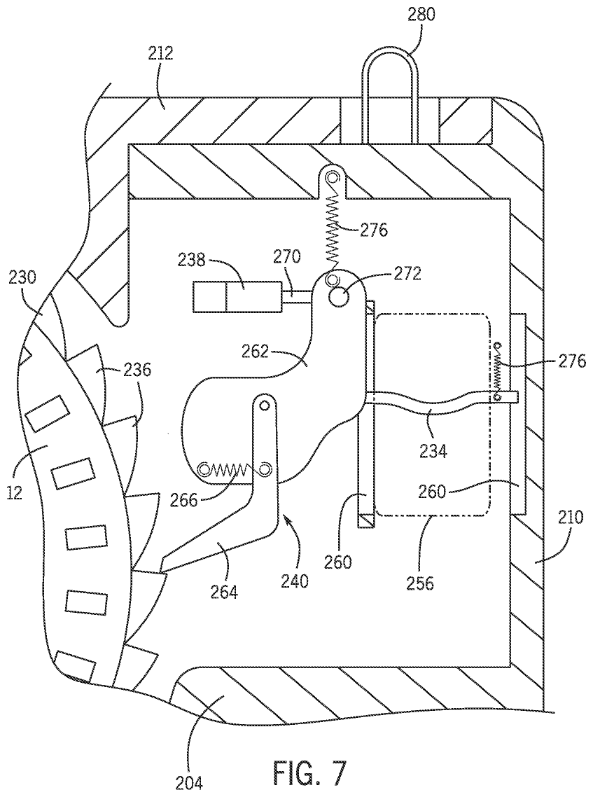

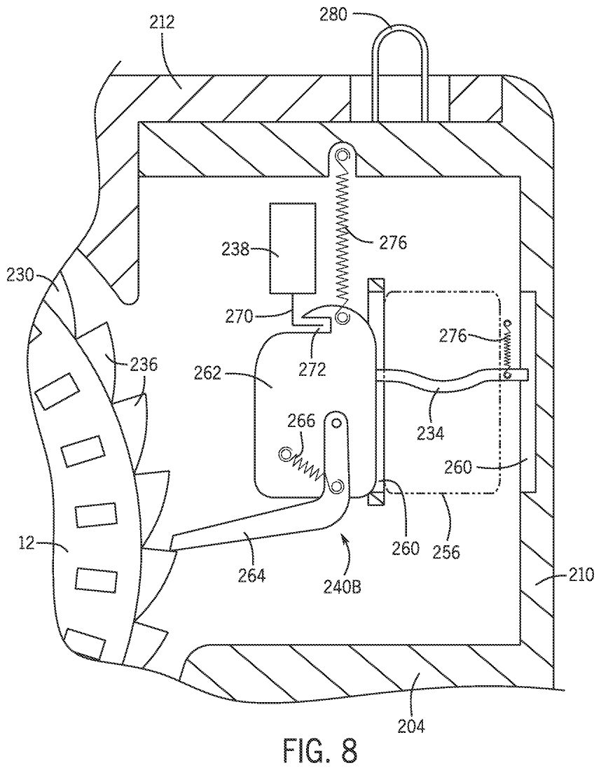

Some embodiments of a medication control device 200 may include a differently configured transmission and locking system. For example, referring to FIG. 8, the transmission 240 includes an electromechanical lock 238 configured to rotate a locking pin 270 rather than move the pin linearly, as in FIGS. 6-7. When moved into the locked position, the locking pin 270 is rotated into engagement with an opening 272, which may be a slot, formed in the connecting member 262, thereby preventing the connecting member 262 from moving.

Further still, some embodiments may include a locking system with redundant locking features. Referring to FIG. 9, the lock 238 includes a first locking pin 270 configured to selectively engage an opening 272 formed in the connecting member 262 and a second locking pin 290 configured to engage one of the locking slots 292 formed around the perimeter of the carousel 230. At least one of the locking pins 270, 290 may be biased into the locked position by a respective pin spring 294. This may be useful, for example, to prevent the advancement of the carousel 230 in the event of a power interruption and/or a failure of the lock motor. The lock motor is operatively linked to the first and second locking pins 270, 290 by a mechanical linkage configured so that the lock 238 can simultaneously move both locking pins 270, 290 between locked and unlock positions with a single lock motor. Additionally or alternatively, some embodiments of the medication control device 200 may include separate locks configured so that movement of the carousel 230, connecting member 262 (or other components of the transmission 240), and/or the trigger 234 can be independently blocked.

In an example of use, the medication control device is provided to the pharmacist, who opens the device and inserts a new blister pack of the medication. The pharmacist closes the medication control device and engages the security tag in a tamper-evident engagement with the door of the medication control device. The medication control device limits the frequency with which the patient is able to access the medication of the prescription for the duration of the prescription, while the security tag provides a tamper-evident indication if the prescription medications are accessed by opening the medication control device. When all of the prescription medication has been consumed, the medication control device is brought back to the pharmacy to be refilled by the pharmacist with a new blister pack of the prescription medication. If the pharmacist notices the tamper-evident security tag is damaged or missing, this may be an indication of medication abuse or misuse and can initiate intervention protocols. One benefit of examples of the medication control device is that even if a single pill dose is accessed early, the timed mechanism limits further access to additional doses. This can prevent overdose by the timed limitation of pill dose access.

Embodiments of the medication control device may be pre-programmed or arranged by the manufacturer to permit access to a pill at regular time intervals (e.g., 3, 6, 12, or 24 hours) since a previous pill was accessed. Still other embodiments may be programmable to change the interval each time that the medication control device is re-loaded with a new blister pack.

Citations to a number of references are made herein. The cited references are incorporated by reference herein in their entireties. In the event that there is an inconsistency between a definition of a term in the specification as compared to a definition of the term in a cited reference, the term should be interpreted based on the definition in the specification.

In the above description, certain terms have been used for brevity, clarity, and understanding. No unnecessary limitations are to be inferred therefrom beyond the requirement of the prior art because such terms are used for descriptive purposes and are intended to be broadly construed. The different systems and method steps described herein may be used alone or in combination with other systems and methods. It is to be expected that various equivalents, alternatives, and modifications are possible within the scope of the appended claims.

This written description uses examples to disclose the invention, including the best mode, and also to enable any person skilled in the art to make and use the invention. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A medication control device for prescription medication in a blister pack, the medication control device comprising:
a housing defining an interior of the housing and a door movable between an open position and a closed position in which the interior of the housing is inaccessible;
a carousel rotatably mounted within the interior of the housing and configured to receive the blister pack;
an actuator configured to advance the carousel to move a blister of the blister pack to a dispensing area of the housing when operated by a user;
a lock configured to selectively inhibit movement of the carousel, the lock configured to selectively move between a locked position in which movement of the carousel is obstructed and an unlocked position in which movement of the carousel is permitted;
a controller configured to, upon detecting advancement of the carousel, start a timer for a predetermined length of time and operate the lock to inhibit movement of the carousel until the predetermined length of time has elapsed; and
a tamper-evident seal configured to retain the door in the closed position until broken, thereby only allowing destructive access to the interior of the housing.

2. The medication control device of claim 1, wherein the predetermined length of time is a minimum dose interval for the prescription medication.

3. The medication control device of claim 1, further comprising a visual indicator, wherein the controller operates the visual indicator to visually present an indication if the predetermined length of time has elapsed.

4. The medication control device of claim 1, further comprising a display device communicatively linked to the controller, wherein the controller is configured to operate the display device to display a remaining amount of time on the timer.

5. The medication control device of claim 1, wherein the lock is configured to engage the carousel with a locking member when the lock is in the locked position to prevent rotation of the carousel.

6. The medication control device of claim 5, wherein the lock member is configured to engage the actuator with a locking member when the lock is in the locked position to prevent operation of the actuator.

7. The medication control device of claim 1, wherein the dispensing area includes a front window formed in a front side of the housing configured to provide access to the blister in the dispensing area for a user to release a dose of medication from the blister; and
a back window formed in a back side of the housing configured to allow the user to retrieve the dose of medication once the dose of medication has been released from the blister.

8. The medication control device of claim 7, wherein the carousel includes a plurality of carousel windows formed around the carousel, wherein the carousel windows correspond to blisters of the blister pack such that the dose of medication passes through a corresponding one of the carousel windows to the back window when the dose of medication is release from the blister.

9. The medication control device of claim 1, further comprising a ratcheting transmission operatively linking the actuator to the carousel, the ratcheting transmission configured to transfer movement of the actuator to rotation of the carousel in a first rotational direction when the actuator is operated by the user.

10. The medication control device of claim 9, wherein the lock is operatively linked to the ratcheting transmission and the lock is controllable, by the controller, to operate the ratcheting transmission to prevent transfer of movement from the actuator to the carousel while the lock is in the locked position.

11. The medication control device of claim 10, wherein the carousel comprises ratcheting teeth formed around a peripheral edge of the carousel and the ratcheting transmission comprises:
a connecting member connected to the actuator such that the connecting member moves between a first position and a second position when the actuator is operated by a user;
a ratcheting arm extending from the connecting member to engage the ratcheting teeth; and
a return spring configured to bias the connecting member into the first position;
wherein engagement between the ratcheting arm and ratcheting teeth causes the carousel to rotate when the connecting member moves from the first position to the second position.

12. The medication control device of claim 11, wherein the ratcheting arm is rotatably coupled to the connecting member and the ratcheting transmission includes an arm return spring configured to urge a distal end of the ratcheting arm into contact with the ratcheting teeth.

13. The medication control device of claim 12, wherein the lock includes a locking member configured to engage an opening formed in the connecting member to prevent movement of the connecting member while the locking member is engaged with the opening; and
wherein the lock is controllable, by the controller, to move the locking member into engagement with the opening to place the lock in the locked position and to disengage the locking member from the opening to place the lock in the unlocked position.

14. The medication control device of claim 1, wherein the actuator is a trigger and housing includes a trigger window formed in at least one of a front side and a back side of the housing, wherein the trigger is accessible to the user only through the trigger window.

15. The medication control device of claim 14, wherein the trigger is slidably mounted on rails positioned within the housing and proximate opposite sides of the trigger window and the user operates the trigger by sliding the trigger on the rails.

16. The medication control device of claim 1, further comprising a locking shackle that extends from one of the housing and the door and is configured to engage a shackle slot on an other one of the housing and the door when the door is in the closed position; and
wherein the tamper-evident seal comprises a tamper evident lock configured to be secured to the lock shackle to retain the door in the closed position.

17. The medication control device of claim 1, wherein the tamper-evident seal comprises a tamper evident tape configured to be adhered to both the housing and the door to retain the door in the closed position.

18. The medication control device of claim 1, further comprising a tamper sensor in communication with the controller and configured to determine if the door has been opened; and
wherein, in response to the tamper sensor determining that the door has been opened, the controller is configured to illuminate an indicator light to indicate that the interior of the housing was accessed.

19. A prescription medication package comprising:
a housing defining an interior of the housing and a door movable between an open position and a closed position in which the interior of the housing is inaccessible;
a blister pack including a plurality of blisters, each blister encapsulating a dose of medication;
a carousel rotatably mounted within the interior of housing and configured to securely receive the blister pack;
a lock configured to selectively inhibit movement of the carousel, the lock configured to selectively move between a locked position in which movement of the carousel is obstructed and an unlocked position in which movement of the carousel is permitted;
a controller configured to, upon detecting advancement of the carousel, start a timer for a predetermined length of time and operate the lock to inhibit movement of the carousel until the predetermined length of time has elapsed; and
a tamper-evident seal configured to retain the door in the closed position until broken, thereby only allowing destructive access to the interior of the housing.
